(19) United States
(12) Patent Application Publication
(10) Pub. No.: US 2026/0335331 A1
(43) Pub. Date: Oct. 29, 2026

(54) ORAL FORMULATION OF DIAMINOPHENOTHIAZINES AND METHODS OF MAKING AND USING THE SAME IN THE TREATMENT AND/OR PREVENTION OF DISEASES

(71) Applicant: Yin Sze LOH, Singapore (SG)

(72) Inventor: Yin Sze LOH, Singapore (SG)

(21) Appl. No.: 19/113,842

(22) PCT Filed: Sep. 20, 2023
(86) PCT No.: PCT/SG2023/050638

§ 371 Date: Mar. 20, 2025

(30) Foreign Application Priority Data
Sep. 21, 2022 (SG) 10202251102T

(51) Int. Cl. A61K 31/5415; A61K 9/00; A61K 47/36; A61P 31/10; A61P 31/14; A61P 31/20

(52) U.S. Cl. CPC A61K 31/5415; A61K 9/0053; A61K 47/36; A61P 31/10; A61P 31/14; A61P 31/20

(57) ABSTRACT

An oral formulation comprising a diaminophenothiazine compound and a mucoadhesive gastro-retentive platform is disclosed, as well as veterinary uses thereof in treatment and or prevention of diseases such as avian wasting diseases. Methods of treating and/or preventing diseases such as avian wasting disease by administering an effective amount of diaminophenothiazine compound in an oral formulation with a mucoadhesive gastro-retentive platform to an animal suffering from the disease such as avian wasting disease. Treated birds were able to recover from previously untreatable diseases.

### REFERENCE TO RELATED APPLICATIONS

This application claims the priority to Singapore patent application No. 10202251102T, filed 21 Sep. 2022, the contents of which are incorporated herein by reference.

### TECHNICAL FIELD

The present disclosure relates generally to oral formulations of diaminophenothiazines for medications and methods of making and using the same in the treatment and/or prevention of veterinary diseases particularly avian diseases.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Birds live and breed in most terrestrial habitats on all continents on earth. Many species migrate annually over great distances. These common long-distance flights increase the spread of avian diseases. In addition to the common spread from migration, infections in Avian species especially occur in enclosed breeding environments. These infections can spread rapidly among many birds housed in an aviary for breeding purposes leading to significant avian morbidity and mortality through wasting. Wasting disease can be a result of enteric peripheral neuropathy where peripheral nerves of the avian proventricular-gastrointestinal tract becomes dysfunctional. Motility and secretory functions of the gastrointestinal system can be impaired due to neuropathy leading to eventual malabsorption of nutrients, starvation, wasting and death. Perforation of the gastrointestinal tract structures can occur due to severe dilation as ingested food particles remain immotile and accumulates in the gastrointestinal tract. Enteric peripheral neuropathy can even lead to central nervous system dysfunction via the gut-brain axis, with the brain being affected eventually. Three of the most common Avian infections that can lead to such dire consequences include: Avian Megalobacteriosis; Avian Bornavirus infection; and Parrot beak and feather disease. Other avian wasting diseases such as Avian Borna virus cause wasting disease in addition to other severe symptoms to the brain as the virus is able to travel through the nervous system via the gastrointestinal peripheral nerve plexus. For most avian diseases one prevention method to contain infection is to cull the entire bird population living within the same enclosure. Where only a few infected birds have been found this is both cruel and expensive.

Avian Megalobacteriosis or Avian Gastric Yeast is caused by the fungus Macrorhabdus ornithogaster. Commonly reported signs of disease include chronic weight loss, apathy, anorexia, vomiting/regurgitation, and the passage of whole or partial seeds in soft, watery droppings. Digested blood may also be seen in the droppings. In the advanced stages of the disease, there may be vomiting of slimy material. Chronic emaciation occurs over a long period of time. The course of the disease may take months and there may be intermittent periods of recovery and relapse. Because of their size, megabacteria can be easily recognized under the microscope so that diagnosis can be made through the identification of megabacteria in wet mounts or stained smears from crop swabs, proventricular scrapings, and droppings. Radiographs may reveal proventricular dilatation as well as an hourglass-like constriction between the proventriculus and ventriculus. Thickening of the proventricular wall can also be seen with contrast (barium) studies.

Elimination of Avian Megalobacteriosis can be difficult, as no effective treatment or prevention is known. Megabacteria have been shown to be resistant to tested antibiotics. In addition, control of the disease has not been adequately described. Treatment protocols that have been described as having some promise attempt to lower the pH of the proventriculus (often with Lactobacillus) to reverse the effects of the disease and make the environment less habitable for the megabacteria. Acidification of the drinking water with hydrochloric acid may help in some cases of megabacteriosis, as well in cases of enteritis that are not responsive to traditional therapy. Some success has been observed with the fungiside amphotericin B, however it can be difficult to obtain oral formulations of amphotericin B.

Avian bornaviral ganglioneuritis, often referred to as parrot wasting disease, is speculated to be caused by a recently discovered avian virus from the taxonomic family Bornaviridae or avian bornavirus (ABV). Research regarding the pathogenesis and treatment for this disease is ongoing. At this time, numerous questions remain unanswered regarding the transmission of the disease, best practices for diagnostic sampling and testing, and whether currently used drug therapies are effective or harmful for afflicted birds. Avian bornavirus is an opportunistic virus that also causes proventricular dilatation disease (PDD) mainly in parrots. PDD is a fatal inflammatory disease of a birds proventriculus in the upper gastrointestinal tract.

Psittacine beak and feather disease (PBFD) is also known as parrot beak and feather disease, psittacine circovirus (PCV) or Psittacine Circoviral Disease (PCD). It is the most common and highly infectious viral disease among psittacines, parrots and related species. caused by a psittacine circovirus. Circovirus infections have been reported in at least 18 species of wild and domestic non-psittaccine birds of the orders Columbiformes, Passeriformes, Anseriformes, Galliformes, Charidiiformes, and Struthioformes, including pigeons, finches, canaries, geese, ducks, pheasants, gulls, and ostriches. It occurs in an acutely fatal form and a chronic form. The disease presents as an immunosuppressive condition with irreversible loss of feathers, beak and claw deformities eventually leading to death often from secondary infections taking hold due to the reduced immunity. Common diagnosis includes histology, PCR or various antibody technologies including ELISA. Currently no commercially viable specific treatment for birds affected with chronic PBFD exists. Detection and culling of infected birds are normally performed in infected captive or commercial flocks as a means of prevention from further spread of the disease. There is an ongoing need to develop a vaccine or treatment or prevention to combat BFDV infection.

Diaminophenothiazine (DAPTZ) compounds eg. Methylene blue have previously been shown to be useful for the management of several human microbial infections including Vaginal candidiasis, Hepatitis C and Malaria.

Non-limiting examples of diaminophenothiazine compounds (reduced form and oxidized form, after oxidation with H₂+HCl) are shown in the following formulas:

Reduced form

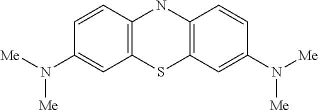 [SMILES: CN(C)c1ccc2c(c1)Sc1cc(N(C)C)ccc1N2]

Oxidized form (MTC)

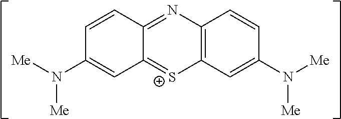

### Cl ⊕

As an example, oxidized and reduced forms of the DAPTZ compounds include, but are not limited to methylthioninium chloride (MTC). MTC give rise to two different forms of active methylthioninium (MT) moieties, the positively charged oxidized form and the neutrally charged reduced (leuco) form of the MT moieties respectively. The MT moieties are the active moieties that are absorbed by the stomach and intestines to enter the blood stream. It is to be appreciated that the oxidized positively charged MT form is blue in colour as compared to the reduced colourless neutrally charged leuco form. As such, these two forms are visually distinctive. DAPTZ compounds are commonly employed as a medication for the treatment of methemoglobinemia. DAPTZ compounds are commonly injected intravenously as oral formulations are short acting.

There exists a need to provide treatment and/or prevention of diseases such as avian wasting and/or alleviate at least one of the aforementioned problems.

### SUMMARY

An oral formulation for a treatment and/or prevention of diseases such as avian wasting having a sustained/controlled release or targeting upper gastrointestinal tract is envisaged.

Accordingly, an aspect of the invention refers to an oral formulation comprising a diaminophenothiazine compound and a mucoadhesive gastro-retentive platform, wherein the diaminophenothiazine compound comprises methylthioninium chloride (MTC) for veterinary use in treatment and/or prevention of a disease.

According to another aspect of the invention refers to an oral formulation comprising a diaminophenothiazine compound and a mucoadhesive gastro-retentive platform for veterinary use in treatment and/or prevention of a disease associated with enteric peripheral neuropathy.

According to another aspect of the invention refers to an oral formulation comprising a diaminophenothiazine compound and a mucoadhesive gastro-retentive platform for veterinary use in treatment and/or prevention of an avian wasting disease.

According to another aspect of the invention refers to an oral formulation comprising a diaminophenothiazine compound and a mucoadhesive gastro-retentive platform for veterinary use in treatment and/or prevention of oesophago-gastric perforations or ulcerations of an upper gastrointestinal tract caused by infective agents.

According to another aspect of the invention refers to use of a diaminophenothiazine compound in the manufacture of an oral formulation with gastro-retentive platform for the treatment and/or prevention of an avian wasting disease.

According to another aspect of the invention refers to a method of treating or preventing a disease comprising: administering an effective amount of diaminophenothiazine compound in an oral formulation with mucoadhesive gastro-retentive platform to a non-human animal suffering from the disease, wherein the diaminophenothiazine compound comprises methylthioninium chloride (MTC).

According to another aspect of the invention refers to a composition comprising a diaminophenothiazine compound, wherein the diaminophenothiazine compound comprises methylthioninium chloride (MTC), and a mucoadhesive gastro-retentive platform, wherein the mucoadhesive gastro-retentive platform is selected from one or more of the groups consisting of one or more polysaccharide, chitosan and hyaluronic acid.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of non-limiting examples only, embodiments of the present invention,

FIG. 1: oral formulation in solution comprising a diaminophenothiazine compound and a mucoadhesive carbohydrate.

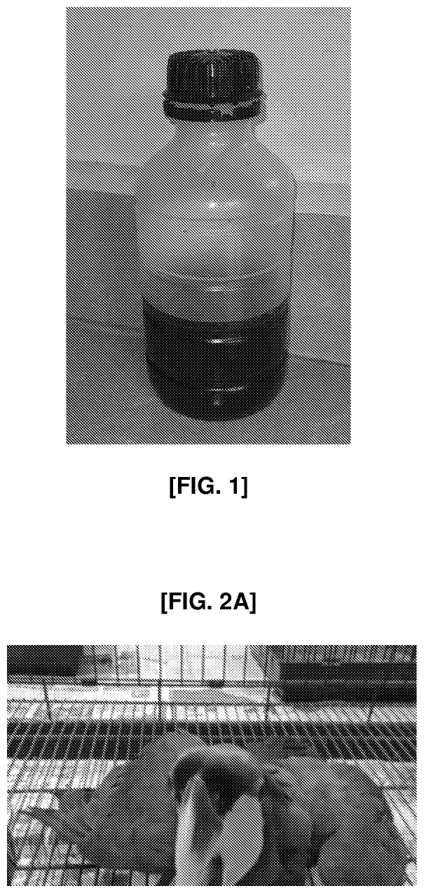

FIG. 2: A bird (A) before and (B) after treatment for avian megalobacteriosis (C) having only 2% of the original megabacteria pathogen load in stool smear microscopy after treatment.

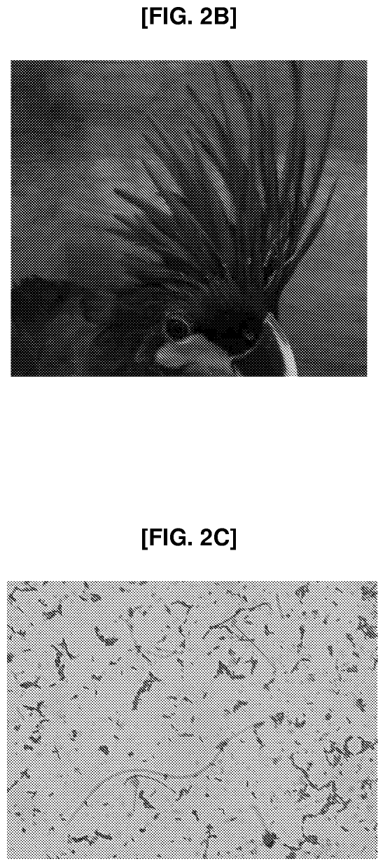

FIG. 3: A schematic of (A) an embodiment of the oral formulation comprising a diaminophenothiazine compound and a mucoadhesive carbohydrate such as a mixture of the diaminophenothiazine compound and a polysaccharide as the mucoadhesive gastro retentive platform that sticks to the stomach walls and (B) illustrates the avian entericperipheral nervous system.

FIG. 4: Weight of black palm cockatoo diagnosed with megabacteria infection over the course of treatment with an oral formulation of MTC and hyaluronic acid.

### DETAILED DESCRIPTION

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the document, unless the context requires otherwise, the word “include” or variations such as “includes” or “including” will be understood to imply the inclusion of a stated integer or group of integers but not the exclusion of any other integer or group of integers.

Throughout the description, it is to be appreciated that the term ‘mucoadhesive gastro retentive platform’ and its plural form can include frameworks that are able to retain an effective amount of diaminophenothiazine compound within the stomach for 20 minute or 1 to 3 hours or more. In various embodiments the mucoadhesive gastro-retentive platform may be referred to or comprise mucoadhesive agent such as a carbohydrate, a protein, a glycoprotein, a polymer or any other mucoadhesive agent with sufficient interfacial forces to anchor to the mucosal lining of the stomach. In various embodiments the mucoadhesive gastro-retentive platform may be referred to or comprise a mucoadhesive carbohydrate as carbohydrates may provide a platform or framework that allows the diaminophenothiazine compound to be retained in the stomach for a longer time allowing increased absorbance, activity and effectiveness. Other mucoadhesive gastro retentive platforms may also be used provided they are able to be retained in the stomach for a longer time allowing the diaminophenothiazine compound to have increased absorbance, activity and effectiveness. In various embodiments the mucoadhesive gastro retentive platform may include frameworks that are able to retain an effective amount of diaminophenothiazine compound within the stomach for 20 minutes or more, 30 minutes or more, 50 minutes or more. In various embodiments the mucoadhesive gastro-retentive platform may include frameworks that are able to retain an effective amount of diaminophenothiazine compound within the stomach for 1 hour or more, 2 hours or more, 3 hours or more, 4 hours or more, 5 hours or more, 6 hours or more. In various embodiments the mucoadhesive gastro-retentive platform may increase bioavailability of an effective amount of diaminophenothiazine compound by more than 1.2 times, 1.3 times, 1.4 times, 1.5 times, 1.6 times, 1.7 times, 1.8 times, 1.9 time or more than 2 times.

As used herein the term ‘veterinary use’ refers to use in all animals except for humans. In various embodiments the non-human animal may be a vertebrate. In various embodiments the non-human animal may be an avian species.

As used herein the term ‘wasting disease’ refers to a disease in a non-human animal caused by an infective agent such as bacteria, fungus or virus that results in weight loss of the non-human animal. In various embodiments the wasting disease comprises avian wasting disease including but not limited to avian megalobacteriosis; avian bornavirus infection; avian paramyxovirus and parrot beak and feather disease.

As used herein the term ‘treating’, ‘treat’ or ‘treatment’ refers to stopping or reducing further sickness, wasting, infection or death in a non-human animal. In various embodiments the non-human animal may be a vertebrate. In various embodiments the non-human animal may be an avian species. In various embodiments the treatment comprises a veterinary treatment. In various embodiments stopping or reducing further sickness, wasting, infection or death in a non-human animal may be achieved by reducing the infective agent such as bacteria, fungus or virus. In various embodiments stopping or reducing further sickness, wasting, infection or death in a non-human animal may be achieved by reducing the presence of Macrorhabdus ornithogaster megabacteria from stool swabs or other samples. In various embodiments stopping or reducing further sickness, wasting, infection or death in a non-human animal may be achieved by reducing the presence of Bornaviridae virus from blood or other samples. In various embodiments stopping or reducing further sickness, wasting, infection or death in a non-human animal may be achieved by reducing the presence of psittacine Circovirus virus from blood or other samples. In various embodiments stopping or reducing further sickness, wasting, infection or death in a non-human animal may be achieved by reducing the presence of Asian Borna virus. In various embodiments stopping or reducing further sickness, wasting, infection in a non-human animal may be measured by maintaining or increasing the weight or mass of the non-human animal. In various embodiments stopping or reducing further sickness, wasting, infection or death in a non-human animal may be achieved by reducing the presence of avian paramyxovirus.

As used herein the term “preventing”, “prevent”, or “prevention” refers to stopping or minimizing non-infected animals who may be immunocompromised or may have been in contact with infected animals from becoming sick, being infected or dying. In various embodiments preventing may be referred to as prophylactic. In various embodiments the non-human animal may be a vertebrate. In various embodiments the non-human animal may be an avian species. In various embodiments the prevention comprises a veterinary use for prevention. In various embodiments the prophylactic or preventative formulation may be given to immunocompromised non-human animals to stop, block or minimize the chance of infection by an infective agent such as bacteria, fungus or virus. In various embodiments the prophylactic or preventative formulation may be given to non-human animals that have been in contact with infected animals in an attempt to stop, block or minimize the chance of infection by an infective agent such as bacteria, fungus or virus. In various embodiments stopping, or blocking, or minimizing, or minimizing the chance of infection in a non-human animal may be achieved by preventing infection by Macrorhabdus ornithogaster. In various embodiments stopping, or blocking, or minimizing, or minimizing the chance of infection in a non-human animal may be achieved by preventing infection by Bornaviridae virus. In various embodiments stopping, or blocking, or minimizing, or minimizing the chance of infection in a non-human animal may be achieved by preventing infection by psittacine Circovirus virus. In various embodiments stopping, or blocking, or minimizing, or minimizing the chance of infection in a non-human animal may be achieved by preventing infection by Asian Borna virus. In various embodiments stopping, or blocking, or minimizing, or minimizing the chance of infection in a non-human animal may be achieved by preventing infection by avian paramyxovirus. It may be understood that the dosing regimen or frequency for prevention may be lower than the dosing regimen or frequency required for treatment. In various embodiments the oral formulation is to be administered weekly for prevention.

Unless defined otherwise, all other technical and scientific terms used herein have the same meaning as is commonly understood by a skilled person to which the subject matter herein belongs.

According to various embodiments, there is an oral formulation comprising a diaminophenothiazine compound and mucoadhesive gastro-retentive platform, wherein the diaminophenothiazine compound comprises methylthioninium chloride (MTC) for veterinary use in the treatment and/or prevention of a disease.

In various embodiments the oral formulation may be prepared as a fluid, preferably a gel or a liquid or a spray. In various embodiments the oral formulation may be prepared as a mucoadhesive solid, preferably a powder, a film or microparticle or nanoparticle. In various embodiments the oral formulation may be prepared as a combination of one or more of a gel, a liquid, a spray, a powder, a film, microparticle and nanoparticle. In various embodiments the oral formulation of any of the preparations listed may be added to the drinking water of the non-human animal.

In various embodiments, the formulation is controlled release resulting in the release of the active ingredient of diaminophenothiazine targeting the upper gastrointestinal tract when used. In addition, the formulation has a gastro-retentive property. These properties can help to optimize controlled gastric absorption of diaminophenothiazine compounds overtime and reduce side effects of the drug on the lower large intestines. These properties can help to reduce dosing frequency of the said oral formulation.

In various embodiments, the diaminophenothiazine compound comprises an active ingredient of a DAPTZ compound. In various embodiments, the diaminophenothiazine compound comprises methylthioninium chloride (MTC). In various embodiments, the diaminophenothiazine compound may comprise methylthioninium chloride (MTC). In various embodiments, the MTC diaminophenothiazine compound comprises 3,7-bis(dimethylamino) phenothiazin-5-ium chloride.

In various embodiments, the diaminophenothiazine compound may comprise a compound similar to MTC where the chloride may be replaced by one or more anionic counter ions to achieve electrical neutrality. Examples of suitable anionic counter ions may be salts including pharmaceutically acceptable salts or a halide comprising —Br; —I or NO₃—. In various embodiments, the diaminophenothiazine compound may comprise a compound similar to MTC wherein at least one, at least two at least three or all methyl groups may be replaced by anyone of: H; unsubstituted aliphatic C₂₋₆alkyl; substituted aliphatic C₂₋₆alkyl; unsubstituted C₃₋₆cycloalkyl; substituted C₃₋₆cycloalkyl; unsubstituted C₆₋₁₀carboaryl; substituted C₆₋₁₀carboaryl; unsubstituted C₅₋₁₀heteroaryl; substituted C₅₋₁₀heteroaryl; unsubstituted C₆₋₁₀carboaryl-C₁₋₄alkyl; substituted C₆₋₁₀carboaryl-C₁₋₄alkyl.

In various embodiments, the gastro-retentive platform may comprise a microparticle wherein the diaminophenothiazine compound is encapsulated in microparticles. In various embodiments the micro-particle comprises a poly (lactide-co-glycolide) (PLGA) microspheres however any microparticle known in the art to be able to provide a slow/sustained release profile would be suitable. In various embodiments the PLGA microspheres comprise a polymer ratio of PLGA (50:50), PLGA (75:25), or PLGA (85:15). Such ratios may be able to modulate the release profile of the diaminophenothiazine compound. In various embodiments, the microparticle is coated with a mucoadhesive agent such as a carbohydrate, a protein, a glycoprotein, a polymer or any mucoadhesive agent with sufficient interfacial forces to anchor the microparticles to the mucosal lining of the stomach. In various embodiments, the microparticle is coated with chitosan. In various embodiments, the microparticle is coated with hyaluronic acid

In various embodiments, the mucoadhesive gastro-retentive platform can include a gastro-targeting platform to ensure the release of the active diaminophenothiazine compound/s happens within the gastric cavity where diaminophenothiazines are preferably absorbed in an acidic environment. In various embodiments, the mucoadhesive gastro-retentive platform may be formed of elements that dissolve in low pH allowing any diaminophenothiazine loaded microparticle embedded within a matrix to be disbursed throughout and held in place within the matrix until it reaches the stomach. The lower pH in the stomach then allows for better absorbance of any released diaminophenothiazine loaded microparticles within the stomach that can then adhere to the stomach and slowly release the MTC.

In various embodiments, the mucoadhesive gastro-retentive platform comprises carbohydrates. In various embodiments, the mucoadhesive gastro-retentive platform comprises one or more polysaccharide. In various embodiments, the mucoadhesive gastro-retentive platform comprises one or more glucosamine. In various embodiments, the one or more polysaccharide comprises hyaluronic acid. In various embodiments, the one or more polysaccharide comprises chitosan. In various embodiments, the one or more polysaccharide comprises a combination of hyaluronic acid and chitosan. In various embodiments, the one or more glucosamine comprises hyaluronic acid. In various embodiments, the one or more glucosamine comprises chitosan. In various embodiments, the one or more glucosamine comprises a combination of hyaluronic acid and chitosan. In various embodiments, the mucoadhesive carbohydrate includes one or more polysaccharide comprising hyaluronic acid.

In various embodiments, the mucoadhesive gastro-retentive platform may comprise chitosan a beta (1-4) linked D glucosamine and N-acetyl-D-glucosamine. It is formed as a polymer of the following disaccharide monomer wherein n may be any number from 2 to 10,000.

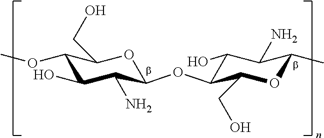

In various embodiments, the mucoadhesive gastro-retentive platform may comprise hyaluronic acid, an anionic, non-sulfated glycosaminoglycan. It may also be called hyaluronan. It is formed as a polymer of the following disaccharide monomer wherein n may be any number from 2 to 20,000.

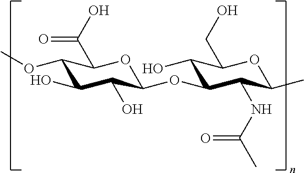

In various embodiments, the mucoadhesive gastro-retentive platform may be selected from one or more of the group consisting of one or more polysaccharides; carbohydrates, microparticles preferably coated with a mucoadhesive agent, protein, polymer and glycoproteins in any of the oral formulations listed herein. In various embodiments, the mucoadhesive gastro-retentive platform comprises a protein such as gelatin. In various embodiments, the mucoadhesive gastro-retentive platform comprises a glycoprotein. In various embodiments, the mucoadhesive gastro-retentive platform comprises a carbohydrate such as chitosan; and hyaluronic acid. In various embodiments, the mucoadhesive gastro-retentive platform comprises one or more polysaccharides. In various embodiments, the mucoadhesive gastro-retentive platform comprises hyaluronic acid. In various embodiments, the mucoadhesive gastro-retentive platform comprises chitosan. In various embodiments, the mucoadhesive gastro-retentive platform comprises a combination of hyaluronic acid and chitosan. In various embodiments, the one or more polysaccharide comprises hyaluronic acid. In various embodiments, the one or more polysaccharide comprises chitosan. In various embodiments, the one or more polysaccharide comprises a combination of hyaluronic acid and chitosan.

Without being limited to any theory the inventor postulates that the hyaluronic acid and/or chitosan forms an attachment to the mucosal lining of the stomach or duodenum wall holding the active ingredient of diaminophenothiazine around this area for an extended time. In various embodiments the carbohydrates with these postulated muco-adhesive properties are better suited for use in avian species as the upper gastrointestinal tract of birds is very compartmentalized with specialized mechanisms to move food through the system consequently avian species have less gastric fluid volume in proportion to surface area of the upper gastrointestinal tract of other animals such as in humans. This means the mucoadhesive does not get stuck in the bird's oral cavity or anywhere in the upper gastrointestinal tract allowing the mucoadhesive gastro-retentive platform to move freely into the stomach or duodenum where it is then able to attach to the mucosal lining of the stomach or duodenum wall.

In various embodiments, the diaminophenothiazine compound comprises methylthioninium chloride (MTC). In various embodiments, the formulation comprises methylthioninium chloride (MTC) and mucoadhesive gastro-retentive platform. In various embodiments, the formulation comprises methylthioninium chloride (MTC) and hyaluronic acid. In various embodiments, the formulation comprises methylthioninium chloride (MTC) and chitosan. In various embodiments, the formulation comprises methylthioninium chloride (MTC) and a mucoadhesive carbohydrate as the mucoadhesive gastro-retentive platform.

In various embodiments, an effective amount of diaminophenothiazine compound is formulated with 2 to 3 times an amount of mucoadhesive gastro-retentive platform. In various embodiments, the formulation comprises 1 part an effective amount of diaminophenothiazine compound to 2 to 3 parts mucoadhesive gastro-retentive platform dissolved in a solvent. In various embodiments the solvent is sterile water. In various embodiments other solvents such as buffered solutions, saline solutions, juice solutions may also be used. In various embodiments, 20 to 50 mg of diaminophenothiazine compound and 60 to 100 mg of mucoadhesive gastro-retentive platform is dissolved in 1 liter of the solvent. In various embodiments, 25 to 45 mg of diaminophenothiazine compound and 65 to 95 mg of mucoadhesive gastro-retentive platform is dissolved in 1 liter of the solvent. In various embodiments, 30 mg of diaminophenothiazine compound and 70 to 90 mg of mucoadhesive gastro-retentive platform is dissolved in 1 liter of the solvent. In various embodiments, 30 mg of an effective amount of diaminophenothiazine compound and 80 mg of mucoadhesive gastro-retentive platform is dissolved in 1 liter of the solvent. In various embodiments the oral formulation consists of only diaminophenothiazine compound and a mucoadhesive carbohydrate both dissolved in a solvent. This will ensure that the active ingredient is delivered to the low pH environment of the stomach and that nothing interferes with the active ingredient or the mucoadhesive carbohydrate in achieving this.

In various embodiments, the formulation further comprises other accompanying active ingredients, additives, excipients, diluents, binders, lubricants, disintegrators, fillers, stabilizers, surfactants, antioxidants, or combinations thereof. In various embodiments, the other accompanying active ingredients may comprise any known antibiotics, anti-inflammatories antifungals, or antivirals.

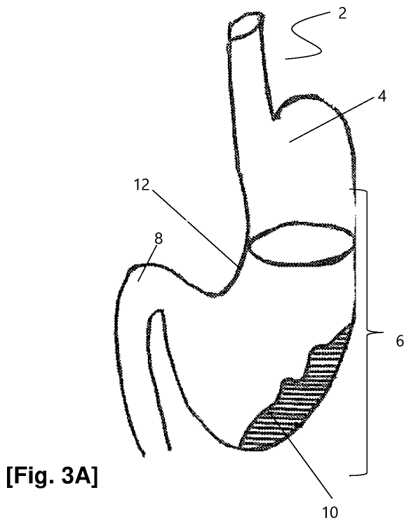

According to various embodiments, there is an oral formulation comprising a diaminophenothiazine compound and mucoadhesive gastro-retentive platform for use in treatment and/or prevention of a disease wherein the disease comprises a disease associated with enteric peripheral neuropathy. In various embodiments infectious agents such as Asian Borna virus, where the peripheral nerves of the avian proventricular-gastrointestinal tract become dysfunctional (See FIG. 3B). In various embodiments infectious agents such as avian paramyxovirus also infect the peripheral nerves of the avian proventricular-gastrointestinal tract depicted in FIG. 3B. Motility and secretory functions of the gastrointestinal system can be impaired due to neuropathy leading to eventual malabsorption of nutrients, starvation, wasting and death. Perforation of the gastrointestinal tract structures can occur due to severe dilation as ingested food particles remain immotile and accumulates in the gastrointestinal tract. Enteric peripheral neuropathy therefore results in and may be referred to as a wasting disease such as an avian wasting disease. In various embodiments, the oral formulation comprises any formulation described herein above. In various embodiments the disease comprises wasting disease. In various embodiments the oral formulation comprising a diaminophenothiazine compound and mucoadhesive gastro-retentive platform is for veterinary use in treatment and/or prevention of a disease associated with enteric peripheral neuropathy.

In various embodiments, the formulation is prepared to be administered daily. In various embodiments, the formulation is prepared to be administered weekly. In various embodiments, the formulation for use in treatment of a disease is prepared to be administered daily. In various embodiments, the formulation for use in prevention of a disease is prepared to be administered weekly.

In various embodiments, the oral formulation comprising a diaminophenothiazine compound and mucoadhesive gastro-retentive platform may be for use in treatment and/or prevention of diseases caused by infective agents such as bacteria, fungi or viruses that effect the stomach or upper gastrointestinal tract. In various embodiments the disease is wasting disease. In various embodiments the oral formulation comprising a diaminophenothiazine compound and mucoadhesive gastro-retentive platform is for veterinary use in treatment and/or prevention of a veterinary wasting disease. In various embodiments the oral formulation comprising a diaminophenothiazine compound and mucoadhesive gastro-retentive platform may be for use in treatment and/or prevention of oesophago-gastric perforations and ulcerations. In various embodiments the oesophago-gastric perforations and ulcerations may be caused by fungi. In various embodiments the oesophago-gastric perforations and ulcerations may be caused by Macrorhabdus ornithogaster.

According to various embodiments, there is an oral formulation comprising a diaminophenothiazine compound and mucoadhesive gastro-retentive platform for use in treatment and/or prevention of an avian wasting disease. In various embodiments, the oral formulation comprises any formulation described herein above.

In various embodiments, the formulation for treatment and/or prevention of an avian wasting disease is prepared to be administered daily. In various embodiments, the formulation for treatment and/or prevention of an avian wasting disease is prepared to be administered weekly. In various embodiments, the formulation for use in treatment of an avian wasting disease is prepared to be administered daily. In various embodiments, the formulation for use in prevention of an avian wasting disease is prepared to be administered weekly. In various embodiments, the formulation for use in prevention of an avian wasting disease is prepared to be administered monthly.

In various embodiments, it is to be appreciated that the term ‘avian wasting disease’ and its plural form include diseases that cause birds to lose weight and in some cases muscle atrophy or poor digestion. In various embodiments, avian wasting disease includes a disease associated with avian enteric peripheral neuropathy, avian megalobacteriosis; avian paramyxovirus, avian bornavirus infection; Asian Borna virus or parrot beak and feather disease.

In various embodiments, the avian wasting disease is selected from avian megalobacteriosis; avian paramyxovirus, avian bornavirus infection; and parrot beak a disease associated with enteric peripheral neuropathy and feather disease.

In various embodiments, the formulation is prepared to be administered to a bird's mouth preferably via oral gavage using a syringe although pipettes, eyedroppers, tube feeding and other suitable delivery methods for oral gavage may be used.

In various embodiments, the disease is a gastrointestinal infection comprising oesophago-gastric perforations and ulcerations caused by infective agents. In various embodiments the wasting disease comprises gastrointestinal infection having oesophago-gastric perforations and ulcerations caused by infective agents.

According to various embodiments, there is an oral formulation comprising a diaminophenothiazine compound and mucoadhesive gastro-retentive platform for use in treatment and/or prevention of oesophago-gastric perforations and ulcerations. In various embodiments, the oral formulation comprises any formulation described herein above. In various embodiments the oesophago-gastric perforations and ulcerations may be caused by fungi. In various embodiments the gastric perforations and ulcerations may be caused by Macrorhabdus ornithogaster.

According to various embodiments, there is use of a diaminophenothiazine compound in the manufacture of an oral formulation with a mucoadhesive gastro-retentive platform for the treatment and/or prevention of a disease. In various embodiments, the oral formulation comprises any formulation described herein above. In various embodiments, the disease is a disease associated with enteric peripheral neuropathy. In various embodiments, the disease is avian wasting disease. In various embodiments, the disease is oesophago-gastric perforations and ulcerations.

In various embodiments, the avian wasting disease is selected from avian megalobacteriosis; avian paramyxovirus, avian bornavirus infection; and parrot beak a disease associated with enteric peripheral neuropathy and feather disease.

In various embodiments, the formulation is prepared to be administered to a bird's mouth preferably via oral gavage using a syringe although pipettes, eyedroppers tube feeding and other suitable delivery methods for oral gavage may be used.

According to various embodiments, there is a method of treating or preventing a disease comprising: administering an effective amount of diaminophenothiazine compound in an oral formulation with mucoadhesive gastro-retentive platform to a non-human animal suffering from the disease wherein the diaminophenothiazine compound comprises methylthioninium chloride (MTC).

In various embodiments the disease is avian wasting disease. In various embodiments the disease is a disease associated with enteric peripheral neuropathy. In various embodiments, the disease is a gastrointestinal infection comprising oesophago-gastric perforations and ulcerations of the upper gastrointestinal tract caused by infective agents.

In various embodiments the non-human animal may be a vertebrate and the disease is associated with enteric peripheral neuropathy. In various embodiments the non-human animal may be a vertebrate and the disease is a wasting disease. In various embodiments the non-human animal may be a vertebrate and the disease is associated with oesophago-gastric perforations and/or ulcerations. In various embodiments the non-human animal may be an avian species and the disease is associated with enteric peripheral neuropathy. In various embodiments the non-human animal may be an avian species and the disease is an avian wasting disease. In various embodiments the disease causes oesophago-gastric perforations and/or ulcerations.

In various embodiments, the diaminophenothiazine compound used in the method comprises methylthioninium chloride (MTC). In various embodiments, the formulation used in the method comprises methylthioninium chloride (MTC) and mucoadhesive gastro-retentive platform. In various embodiments, the formulation used in the method comprises methylthioninium chloride (MTC) and hyaluronic acid. In various embodiments, the formulation used in the method comprises methylthioninium chloride (MTC) and chitosan.

In various embodiments, the effective amount of diaminophenothiazine compound with 2 to 3 times an amount of mucoadhesive gastro-retentive platform is formulated for use as the oral formulation for administration. In various embodiments, the oral formulation used in the method comprises 1 part an effective amount of diaminophenothiazine compound to 2 to 3 parts mucoadhesive gastro-retentive platform dissolved in a solvent. In various embodiments used in the method the solvent may comprise buffered solutions, saline solutions, juice solutions, water solutions or any other solutions known in the art to be suitable for oral consumption. In various embodiments used in the method the solvent is sterile water. In various embodiments, the oral formulation used in the method comprises 20 to 50 mg of diaminophenothiazine compound and 60 to 100 mg of mucoadhesive gastro-retentive platform is dissolved in 1 liter of the solvent. In various embodiments, the oral formulation used in the method comprises 25 to 45 mg of diaminophenothiazine compound and 65 to 95 mg of mucoadhesive gastro-retentive platform is dissolved in 1 liter of the solvent. In various embodiments, the oral formulation used in the method comprises 30 mg of diaminophenothiazine compound and 70 to 90 mg of mucoadhesive gastro-retentive platform is dissolved in 1 liter of the solvent. In various embodiments, the oral formulation used in the method comprises 30 mg of diaminophenothiazine compound and 80 mg of mucoadhesive gastro-retentive platform is dissolved in 1 liter of the solvent.

In various embodiments, the effective amount of diaminophenothiazine compound in an oral formulation with mucoadhesive gastro-retentive platform is administered daily for the method of treatment. In various embodiments, the oral formulation comprises any formulation described herein above. In various embodiments, less than 5 μg of diaminophenothiazine compound is administered daily per kilogram of the weight of the non-human animal. In various embodiments, less than 4.5 μg of diaminophenothiazine compound is administered daily per kilogram of the weight of the non-human animal. In various embodiments, less than 4 μg of diaminophenothiazine compound is administered daily per kilogram of the weight of the non-human animal.

In various embodiments, the effective amount of diaminophenothiazine compound in an oral formulation with mucoadhesive gastro-retentive platform is administered weekly for the method of prevention. In various embodiments, the oral formulation comprises any formulation described herein above. In various embodiments, less than 5 μg of diaminophenothiazine compound is administered weekly per kilogram of the weight of the non-human animal. In various embodiments, less than 4.5 μg of diaminophenothiazine compound is administered weekly per kilogram of the weight of the non-human animal. In various embodiments, less than 4 μg of diaminophenothiazine compound is administered weekly per kilogram of the weight of the non-human animal.

In various embodiments, the method of treating an avian wasting disease is for treating avian megalobacteriosis.

In various embodiments, the method further comprises determining whether Macrorhabdus ornithogaster is present in the bird suffering from avian wasting disease or avian megalobacteriosis after receiving a daily dose of the oral formulation for at least one month. In various embodiments determining whether Macrorhabdus ornithogaster is present is made in a stool smear under the microscope.

In various embodiments, the method of treating an avian wasting disease is for treating avian bornavirus infection.

In various embodiments, the method further comprises determining whether bornavirus is present in the bird suffering from avian wasting disease or bornavirus infection after receiving a daily dose of the oral formulation for at least one month. In various embodiments, determining whether bornavirus is present is determined using PCR of a cloacal smear.

In various embodiments, the method of treating an avian wasting disease is for treating parrot beak and feather disease.

In various embodiments, the method further comprises determining whether psittacine circovirus is present in the bird suffering from avian wasting disease or parrot beak and feather disease after receiving a daily dose of the oral formulation for at least one month. In various embodiments determining whether circovirus is present is determined using PCR on a blood test.

In various embodiments, the method of treating an avian wasting disease is for treating Asian borna virus infection.

In various embodiments, the method further comprises determining whether Asian borna virus is present in the bird suffering from avian wasting disease or Asian borna virus infection after receiving a daily dose of the oral formulation for at least one month. In various embodiments, determining whether Asian borna virus is present is determined using PCR of a cloacal smear.

In various embodiments, the method is for treating the gastric perforations and ulcerations. In various embodiments, the method is for treating avian megalobacteriosis.

In various embodiments, the method further comprising determining whether Macrorhabdus ornithogaster is present in the bird suffering from the avian wasting disease after receiving a daily dose of the oral formulation for at least one month. In various embodiments determining whether Macrorhabdus ornithogaster is present is made in a stool smear under the microscope.

In various embodiments, the method is for treating gastric perforations and ulcerations.

In various embodiments, the mucoadhesive gastro-retentive platform comprises a carbohydrate used in the method, wherein the carbohydrate is selected from one or more of the groups consisting of: one or more polysaccharide; glycoprotein chitosan; and hyaluronic acid. In various embodiments, the mucoadhesive gastro-retentive platform used in the method comprises any one of the embodiments of mucoadhesive gastro-retentive platform listed herein above.

When an oral formulation comprising a diaminophenothiazine compound and a mucoadhesive gastro-retentive platform is used, the active ingredient MTC is able to remain in the fundus or the body of the stomach for longer, being able to remain in the acidic environment of the gastric fluid for a longer time permitting better absorption of the active diaminophenothiazine compound. For example, in various embodiments depicted in [FIG. 3] where a mixture 10 of the diaminophenothiazine compound and a mucoadhesive gastro-retentive platform, the mucoadhesive gastro-retentive platform allows the mixture 10 to stick to the side of the stomach 2 in the body of the stomach section 6 enhancing absorption. In various other embodiments, this allows the diaminophenothiazine compound to be absorbed in the gastric fluid 12 that has a lower pH, the formulation 10 is able to remain in the stomach 12 either near the fundus 4 or in the body of the stomach 6 for a longer period of time enhancing absorption.

According to various embodiments, there is a composition comprising a diaminophenothiazine compound, wherein the diaminophenothiazine compound comprises methylthioninium chloride (MTC) and a mucoadhesive gastro-retentive platform, wherein the mucoadhesive gastro-retentive platform is selected from one or more of the groups consisting of one or more polysaccharide, chitosan and hyaluronic acid.

In various embodiments the composition further comprises other accompanying ingredients, additives, excipients, diluents, binders, lubricants, disintegrators, fillers, stabilizers, surfactants, antioxidants, or combinations thereof. In various embodiments the compound is formulated for oral delivery preferably for oral delivery to a bird or an avian species.

### EXAMPLES

### Example 1: Treatment of Avian Megalobacteriosis

A black palm cockatoo was diagnosed by a vet with megabacteria infection. The bird was pale, crop was distended, stools were liquid and contained undigested food. This resulted in weight loss of 800 mg from pre-morbid conditions. Given the poor condition the bird was prescribed with palliative supplement management (Papaya seed, juice, formula, and supplements).

The bird was treated for 54 days with a daily dose of 0.05 ml of oral formulation (0.003% MTC and 0.008% hyaluronic acid in sterile water).

TABLE 1
black palm cockatoo's condition over the course of treatment.

| | TREATMENT | WEIGHT | |
|---|---|---|---|
| DATE | DAY | (grams) | CONDITION |
| 4 Mar. 2022 | Pre-treatment | 620 | pale, crop was distended, stools were |
| | (supplement | | liquid and contained undigested food |
| | management) | | |
| 19 Mar. 2022 | Pre-treatment | 615 | Pallor increase, more active |
| | (supplement | | |
| | management) | | |
| 23 Mar. 2022 | Pre-treatment | 635 | Melaena (dark black, tarry feces) |
| | (supplement | | |
| | management) | | |
| 24 Mar. 2022 | 1 | 635 | Melaena |
| 26 Mar. 2022 | 3 | 700 | No more melaena, diarrhea, appetite |
| | | | improved |
| 27 Mar. 2022 | 4 | 735 | Good appetite, well-formed stools |
| 28 Mar. 2022 | 5 | 760 | Good appetite, well-formed stools |
| 30 Mar. 2022 | 7 | 765 | Change to increase seeds in diet |
| 6 Apr. 2022 | 13 | 755 | Very active and well |
| 18 Apr. 2022 | 25 | 735 | Very well and increased activity, |
| | | | proventriculus no longer dilated |
| 28 Apr. 2022 | 35 | 750 | Healthy appetite, weight gain, energetic |
| 4 May 2022 | 41 | 760 | Healthy appetite, weight gain, energetic |
| 12 May 2022 | 49 | 775 | Healthy appetite, weight gain, energetic |
| 17 May 2022 | 54 (last day) | 775 | Healthy appetite, weight gain, energetic |
| 24 May 2022 | Post | 785 | Healthy appetite, weight gain, energetic |
| | treatment | | |
| 25 May 2022 | Post | 790 | Detection of 2% megabacteria in stool |
| | treatment | | smear microscopy |
| 7 Jun. 2022 | Post | 800 | Well and thriving |
| | treatment | | |
| 20 Jun. 2022 | Post | 810 | Remains well and thriving |
| | treatment | | |

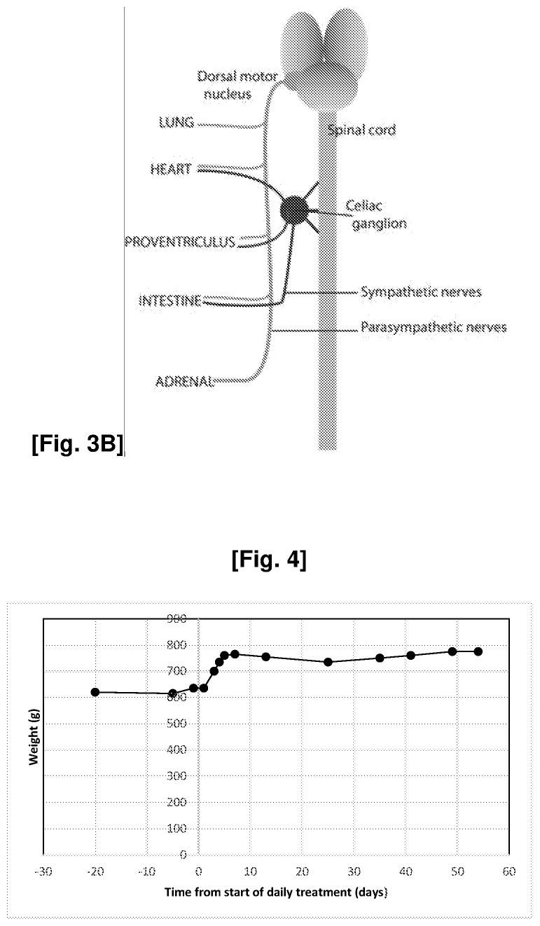

The same treatment has since been successfully repeated in other birds (see [FIG. 4]).

### Example 2: Treatment of Avian Bornavirus Infection

A Moluccan cockatoo presented with regurgitation, poor appetite, indigestion and lack of energy. A PCR test on a cloacal smear tested positive for avian bornavirus (ABV).

The bird was treated for 43 days with a daily dose of 0.15 ml of oral formulation (0.003% MTC and 0.008% hyaluronic acid in sterile water) each morning.

TABLE 2
Moluccan cockatoo's condition over the course of treatment.

| | TREATMENT | |
|---|---|---|
| DATE | DAY | CONDITION |
| 30 Mar. 2022 | Pre-treatment | regurgitation, poor appetite, indigestion |
| | | and lack of energy |
| 9 Jun. 2022 | 1 | poor appetite, lack of energy, |
| | | indigestion |
| 12 Jun. 2022 | 3 | Improved with less indigestion, more |
| | | energetic |
| 21 Jul. 2022 | 43 (last day) | Healthy appetite, weight gain, energetic |
| 18 Aug. 2022 | Post | Negative for PCR detection of ABV in |
| | treatment | cloacal smear |

The same treatment has since been successfully repeated in other birds (data not shown).

### Example 3: Treatment of Parrot Beak and Feather Disease

A Moluccan cockatoo presented with regurgitation, poor appetite, indigestion and lack of energy. The bird was diagnosed with a positive PCR test on a blood sample for psittacine circovirus (PCV) that causes parrot beak and feather disease.

The bird was treated for 43 days with a daily dose of 0.15 ml of oral formulation (0.003% MTC and 0.008% hyaluronic acid in sterile water) each morning.

TABLE 3
Moluccan cockatoo's condition over the course of treatment.

| | TREATMENT | |
|---|---|---|
| DATE | DAY | CONDITION |
| 30 Mar. 2022 | Pre-treatment | regurgitation, poor appetite, indigestion |
| | | and lack of energy |
| 9 Jun. 2022 | 1 | poor appetite, lack of energy, |
| | | indigestion |
| 12 Jun. 2022 | 3 | Improved with less indigestion, more |
| | | energetic |
| 21 Jul. 2022 | 43 (last day) | Healthy appetite, weight gain, energetic |
| 11 Aug. 2022 | Post | Negative for PCR detection of PCV in a |
| | treatment | blood sample |

The same treatment has since been successfully repeated in other birds (data not shown.

A trial has been initiated for treatment of wild water ducks who have been reported to die by the hundreds once PCV infection spreads throughout a flock. Due to the large number of birds and that there are a mix of potentially diseased birds and co-habiting potentially non-diseased birds in such flocks the trial will be carried out in 3 separate flocks with 3 separate dosage regimens: the first is a daily dose; the second is a dose every 3 days and the third is a weekly dose. Concurrently, an untreated flock will be observed.

### Example 4: Prevention of Avian Megalobacteriosis

The black palm cockatoo diagnosed by a vet with megabacteria infection in Example 1 was co-habiting an area with 1 other cockatoo within the same cage. The infected bird had a high amount of megabacteria in its stool meaning the co-habiting bird had a high chance of coming into contact with the infection and being infected by Macrorhabdus ornithogaster. It was initially suggested that the co-habiting bird should be culled to prevent any further spread of the megabacteria infection. As an alternative, the bird was administered a weekly dose of 0.05 ml of oral formulation (0.003% MTC and 0.008% hyaluronic acid in sterile water) until the estimated incubation period of the pathogen was over and the infective source has been resolved.

As a result of the preventative weekly prophylactic the co-habiting bird did not show any signs of infection and remained healthy.

TABLE 4
co-habiting cockatoo's condition over
the course of preventative treatment.

| | TREATMENT | |
|---|---|---|
| DATE | DAY | CONDITION |
| 23 Mar. 2022 | Pre-treatment | Co-habiting partner of infected |
| | | Black palm cockatoo. Remains |
| | | Well and asymptomatic |
| 24 Mar. 2022 | 1 | Remains Well and asymptomatic |
| 26 Mar. 2022 | 3 | Remains Well and asymptomatic |
| 26 May 2022 | Last day of | Remains Well and asymptomatic |
| | treatment | |

The same treatment has since been successfully repeated in other co-habiting birds (data not shown).

It should be further appreciated by the person skilled in the art that variations and combinations of features described above, not being alternatives or substitutes, may be combined to form yet further embodiments falling within the intended scope of the invention.

As would be understood by a person skilled in the art, each embodiment, may be used in combination with other embodiment or several embodiments.

1-24. (canceled)

25. A method of treating or preventing a disease comprising:
administering an effective amount of diaminophenothiazine compound in an oral formulation with a mucoadhesive gastro-retentive platform to a non-human animal suffering from a veterinary disease, wherein the diaminophenothiazine compound comprises methylthioninium chloride (MTC).

26. The method according to claim 25, comprising formulating the effective amount of diaminophenothiazine compound with 2 to 3 times an amount of a mucoadhesive gastro-retentive platform.

27. The method according to claim 25, wherein the mucoadhesive gastro-retentive platform is selected from one or more of the group consisting of a carbohydrate, one or more polysaccharide, chitosan and hyaluronic acid.

28. The method according to claim 25, wherein the effective amount of diaminophenothiazine compound in an oral formulation with a mucoadhesive gastro-retentive platform is administered daily.

29. The method according to claim 25, wherein the disease is an avian wasting disease comprising avian megalobacteriosis.

30. The method according to claim 29, further comprising determining whether Macrorhabdus ornithogaster is present in the bird suffering from avian megalobacteriosis after receiving a daily dose of the oral formulation for at least one month.

31. The method according to claim 25, wherein the disease is an avian wasting disease and/or a disease associated with enteric peripheral neuropathy comprising avian bornavirus infection.

32. The method according to claim 31, further comprising determining whether bornavirus is present in the bird suffering from avian bornavirus infection after receiving a daily dose of the oral formulation for at least one month.

33. The method according to claim 25, wherein the disease is an avian wasting disease comprising parrot beak and feather disease.

34. The method according to claim 33, further comprising determining whether circovirus is present in the bird suffering from parrot beak and feather after receiving a daily dose of the oral formulation for at least one month.

35. The method according to claim 25, wherein the effective amount of diaminophenothiazine compound in an oral formulation with a mucoadhesive gastro-retentive platform is administered weekly.

36. A composition comprising a diaminophenothiazine compound, wherein the diaminophenothiazine compound comprises methylthioninium chloride (MTC) and a mucoadhesive gastro-retentive platform, wherein the mucoadhesive gastro-retentive platform is selected from one or more of the groups consisting of one or more polysaccharide, chitosan and hyaluronic acid.

37. The composition according to claim 36, further comprising other accompanying ingredients, additives, excipients, diluents, binders, lubricants, disintegrators, fillers, stabilizers, surfactants, antioxidants, or combinations thereof.

38. The composition according to claim 36, formulated for oral delivery.
